(19) United States
(12) Patent Application Publication
(10) Pub. No.: US 2012/0116345 A1
(43) Pub. Date: May 10, 2012

(54) Urine-free underwear

(76) Inventor: Joel Dime, Los Angeles, CA (US)

(21) Appl. No.: 12/927,045

(22) Filed: Nov. 5, 2010

(51) Int. Cl. A61F 13/15; A41B 9/04; A41B 9/02

(52) U.S. Cl. 604/396; 2/403; 2/406

(57) ABSTRACT

An underwear garment for wearing on lower torso of a wearer includes an underwear fabric, a waistband, a liquid controlling layer and means for securing the liquid controlling layer onto the inner side of the underwear fabric. The underwear fabric defines a buttocks covering section, a hip covering section, a bottom covering section, a front covering section, and a pair of leg holes. The waistband is provided on a top edge portion of the underwear fabric. The liquid controlling layer is provided on an inner side of the underwear fabric on at least one of the bottom covering section and the front covering section of the underwear fabric for blocking liquid to come into contact with the front covering section of the underwear fabric.

### BACKGROUND OF THE PRESENT INVENTION

1. Field of Invention

The present invention relates to underwear, and more particularly to an underwear garment comprising a liquid controlling layer which is capable of blocking or absorbing liquid which accidentally or undesirably comes into contact with the liquid controlling layer.

2. Description of Related Arts

A conventional underwear garment, such as an underwear garment for men, usually comprises an underwear fabric having a buttock covering portion, a hip portion portion, a bottom covering portion a front covering section and a pair of leg holes, a waistband provided on a top edge portion of the underwear fabric for providing a predetermined elasticity to retain the underwear garment onto the wearer's body when the underwear garment is in use.

Almost invariably, the above-mentioned underwear garment further has an opening formed on the front covering section of the underwear fabric for allowing a wearer to expose his genital (i.e. penis) out of the underwear garment for urination. The provision of the opening allows the wearer to urinate conveniently (especially in public toilets) but this also brings other problems to the use of the underwear garment. More specifically, after the wearer has urinated, urine residual may fall onto the underwear garment and this presents a potentially hygienic problem.

This is especially true when the wearers are the elderly or those having diseases which make them difficult to control urination. For example, for a wearer having a disease which makes him or her difficult to control urination, a small amount of urine may accidentally or undesirably be in contact with the underwear garment. This also presents a potentially hygienic problem for the wearer.

Moreover, when urine residual comes into contact with the underwear fabric, the urine residual may also be absorbed by external clothing of the wearer, such as the wearer's pant, so that urine stains may be formed on the external clothing. This presents great embarrassment to the wearer.

The same problem persists both for men and for women. Although women generally need to take off her underwear garment for urination, when she has the similar kind of disease described above, the hygienic problem discussed above will also be present for women. Thus, traditional underwear garment has not tackled this deep-seated problem and a novel and unobvious underwear must be developed.

### SUMMARY OF THE PRESENT INVENTION

The invention is advantageous in that it provides a urine-free underwear garment comprising a liquid controlling layer which is capable of blocking or absorbing liquid which accidentally or undesirably comes into contact with the liquid controlling layer. Moreover, the underwear garment can be made suitable for men and for women. As a result, the above-mentioned problems for conventional underwear will be tackled.

Another advantage of the invention is to provide a urine-free underwear garment, wherein the liquid controlling layer can be made disposable and replaceable for allowing the wearer to maintain the highest hygienic condition for his or her underwear garment.

Another advantage of the invention is to provide a urine-free underwear garment, wherein the liquid controlling layer does not substantially alter the original structure of that underwear garment, so that the manufacturing of the present invention will be kept to the minimum.

Another advantage of the invention is to provide a urine-free underwear garment, wherein when the when the liquid controlling layer is capable of absorning liquid, it will not affect the air permeability of the remaining portions of the underwear garment. In other words, the liquid controlling layer does not affect the original performance of the underwear garment.

Additional advantages and features of the invention will become apparent from the description which follows, and may be realized by means of the instrumentalities and combinations particular point out in the appended claims.

According to the present invention, the foregoing and other objects and advantages are attained by providing an underwear garment for wearing on lower torso of a wearer, comprising:

an underwear fabric defining a buttocks covering section, a hip covering section, a bottom covering section, a front covering section, and a pair of leg holes;

a waistband provided on a top edge portion of the underwear fabric;

a liquid controlling layer provided on an inner side of the underwear fabric on at least one of the bottom covering section and the front covering section of the underwear fabric near a genital organ of the wearer for blocking liquid coming into contact with the underwear fabric from the genital organ of the wearer; and

means for securing the liquid controlling layer onto the inner side of the underwear fabric.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 is a front view of an underwear garment according to a first preferred embodiment of the present invention.

FIG. 2 is a schematic diagram of the underwear garment according to the above first preferred embodiment of the present invention.

FIG. 3 is a sectional side view of the underwear garment according to the above first preferred embodiment of the present invention.

FIG. 4 is a first alternative mode of the underwear garment according to the above first preferred embodiment of the present invention.

FIG. 5 is a second alternative mode of the underwear garment according to the above first preferred embodiment of the present invention.

FIG. 6 is a schematic diagram of an underwear garment according to a second preferred embodiment of the present invention.

FIG. 7 is another schematic diagram of the underwear garment according to the above second preferred embodiment of the present invention.

FIG. 8 is a sectional side view of the underwear garment according to the above second preferred embodiment of the present invention.

FIG. 9 is a first alternative mode of the underwear garment according to the above second preferred embodiment of the present invention.

FIG. 10 is a second alternative mode of the underwear garment according to the above second preferred embodiment of the present invention.

### DETAILED DESCRIPTION OF THE PREFERRED EMBODIMENT

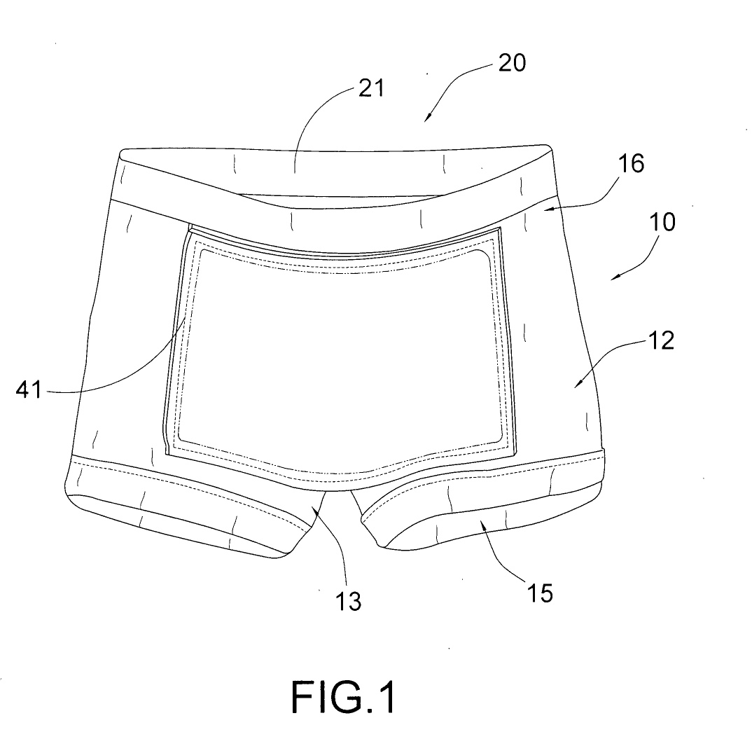

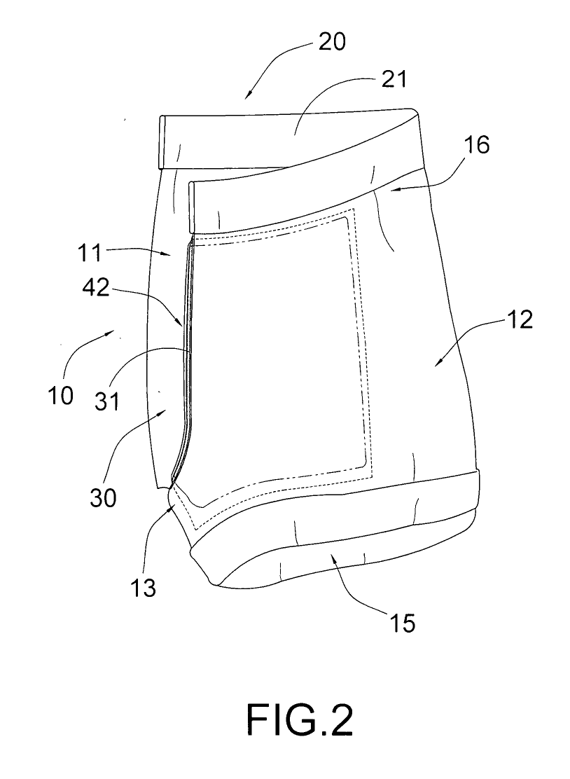

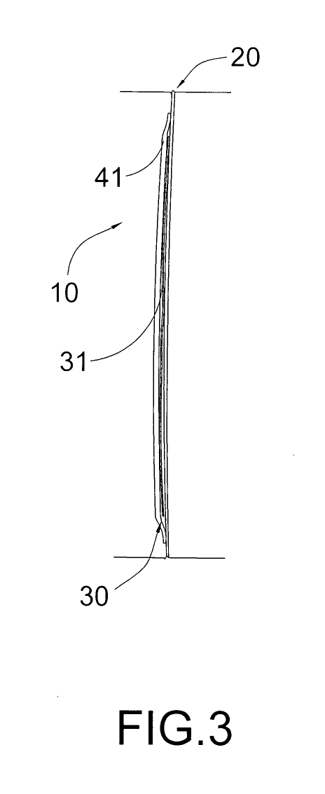

Referring to FIG. 1 to FIG. 3 of the drawings, a urine-free underwear such as an underwear garment for wearing on lower torso of a wearer according to a first preferred embodiment of the present invention is illustrated, in which the underwear garment comprises an underwear fabric 10, a waistband 20, and a liquid controlling layer 30.

The underwear fabric 10 defines a buttock covering section 11, a hip covering section 12, a bottom covering section 13, a front covering section 14, and a pair of leg holes 15 for allowing two legs of the wearer to pass therethrough. On the other hand, the waistband 20 is provided on a top edge portion 16 of the underwear fabric 10. Each of the bottom covering section 13 and the front covering section 14 is genital covering section which is defined as the sections of the underwear fabric 10 which is in the vicinity of the genital of the wearer.

The liquid controlling layer 30 is provided on an inner side of the underwear fabric 10 on at least one of the bottom covering section 13 and the front covering section 14 of the underwear fabric 10 for blocking liquid coming into contact with the front covering section 14 of the underwear fabric 10. The underwear garment further comprises means for securing the liquid controlling layer 30 onto the inner side of the underwear fabric 10 (referred to securing means below).

According to the preferred embodiment of the present invention, the underwear fabric 10 is made of regular fabric material used for conventional underwear garment, such as an underwear pant for men. The underwear garment can also be embodied as boxers or any other forms of underwear for wearing on a lower torso of a wearer, who can be a man or a woman. The preferred embodiment of the present invention is to design the underwear garment for a man as a wearer.

The means for securing the liquid controlling layer 30 onto the inner side of the underwear fabric 10 comprises an inner pocket layer 41 attached on the front covering section 14 of the underwear fabric 10 to define a receiving pocket 42 between the inner pocket layer 41 and the front covering section 14 of the underwear fabric 10. The inner pocket layer 41 is shaped and sized to correspond to a shape and size of the front covering section 14 so that the inner pocket layer 41 is in direct contact with the genital (i.e. the penis) of the wearer. The liquid controlling layer 30 is fittedly received into the receiving pocket 42 so that when liquid, such as urine, comes into contact with the inner pocket layer 41, the liquid will penetrate through the inner pocket layer 41 and will be in contact with the liquid controlling layer 30. The liquid controlling layer 30 will then block the liquid (i.e. the urine) from penetrating the liquid controlling layer 30 so as to avoid the urine residual from coming into contact with the front covering section 14 or even the external clothing of the wearer. As such, the present invention avoids the problem of causing embarrassment to the wearer of traditional underwear.

The waistband 20 comprises an elastic element 21 peripherally formed along a top edge portion of the underwear fabric 10 for allowing the waistband 20 to be fittedly worn onto the wearer's lower torso.

The liquid controlling layer 30 preferably comprises a liquid impermeable layer 31 which has a physical property of blocking liquid from penetrating through the liquid blocking layer 31 so as to avoid urine residual from reaching the external clothing of the wearer.

The liquid controlling layer 30 preferably comprises a fabric layer 31 consisting of a predetermined kind of fibers, and a predetermined amount of absorbent chemicals formed on the fabric layer 31 for absorbing liquid (such as urine of the wearer) coming into contact with the liquid controlling layer 30 through the inner pocket layer 41.

The operation of the present invention is as follows: the liquid controlling layer 30 is received in the receiving pocket 42 of the securing means. If the wearer has difficulty to control urine discharge due to some diseases or other reasons, the urine coming out from the wearer's penis will still pass through the inner pocket layer 41 and come into contact with the liquid controlling layer 30. However, the liquid controlling layer 30 will then block the urine and prevents it from reaching the front covering section 14 of the underwear fabric 10 and the external clothing of the wearer. As a result, the front covering section 14 of the underwear fabric and the external clothing of the wearer will be kept dry so as to induce embarrassment on the part of the wearer. After use, the wearer may wash the entire underwear garment of the present invention for reuse.

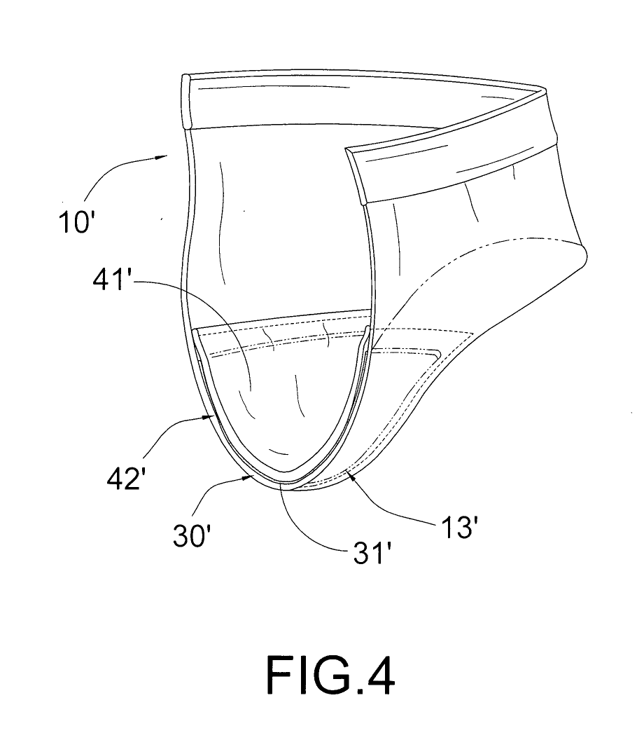

Referring to FIG. 4 of the drawings, a first alternative mode of the urine-free underwear according to a preferred embodiment of the present invention is illustrated. The first alternative mode is similar to the preferred embodiment described above, except that the underwear garment is meant to be worn by a female wearer so that the positions of the liquid controlling layer 30′ and the securing means are different.

According to the first alternative mode, the means for securing the liquid controlling layer 30′ onto the inner side of the underwear fabric. 10′ comprises an inner pocket layer 41′ attached on the bottom covering section 13′ of the underwear fabric 10′ to define a receiving pocket 42′ between the inner pocket layer 41′ and the bottom covering section 13′ of the underwear fabric 10′. The inner pocket layer 41′ is shaped and sized to correspond to a shape and size of the bottom covering section 13′ for fitting female urethra structure. The fabric layer 31′ of the liquid controlling layer 30′ is fittedly received into the receiving pocket 42′ so that when liquid, such as urine, comes into contact with the inner pocket layer 41′, the liquid will penetrate through the inner pocket layer 41′ and will be in contact with the liquid controlling layer 30′. The liquid controlling layer 30′ will then trap the liquid (i.e. the urine) within the liquid controlling layer 30′ so as to keep the inner pocket layer 41′ dry and maintain good hygienic condition for the wearer.

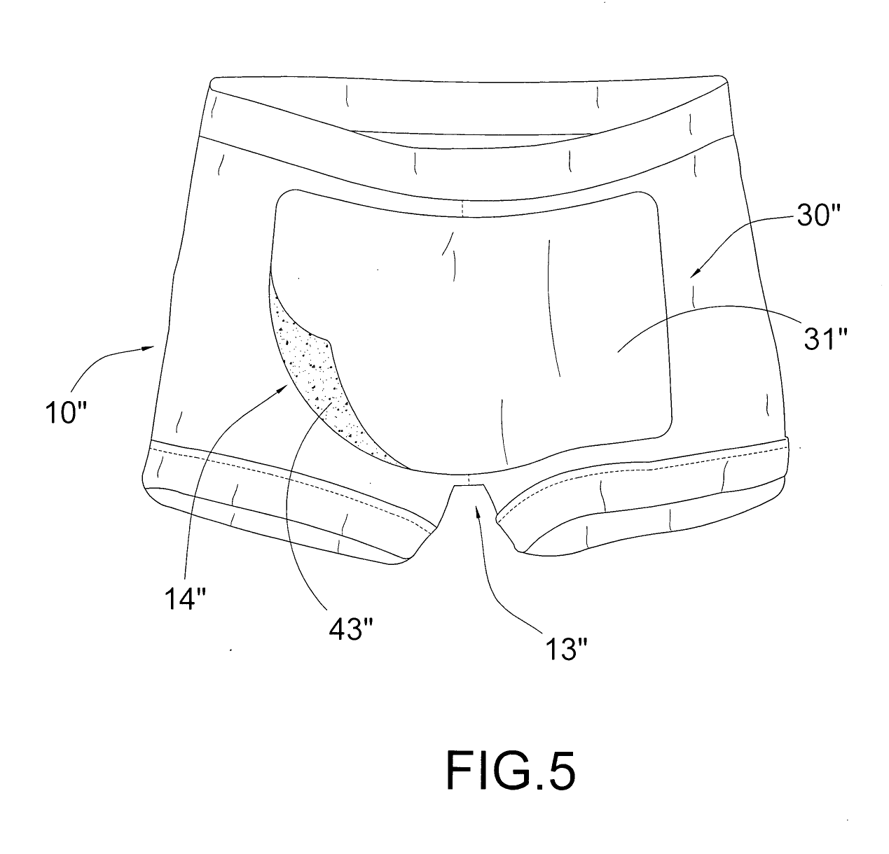

Referring to FIG. 5 of the drawings, a second alternative mode of the urine-free underwear according to the preferred embodiment of the present invention is illustrated. The second alternative mode is similar to the preferred embodiment except the means for securing the liquid controlling layer 30″ onto the inner side of the underwear fabric 10″. According to the second alternative mode, the securing means comprises an adhesive layer 43″ applied on the liquid controlling layer 30″ so as to allow the liquid controlling layer 30″ to be directly and detachably attached onto the front covering section 14″ of the underwear fabric 10″. In other words, the liquid controlling layer 30″ is not protected by the receiving pocket 42. Rather, the liquid controlling layer 30″ is directly but detachably attached onto the inner side of the front covering section 14″ of the underwear fabric 10″. When the urine-free underwear according to this second alternative mode is for female's use, the liquid controlling layer 30″ is detachably attached onto the bottom covering section 13″ (13′) of the underwear fabric 10″ so as to accommodate female's biological structure.

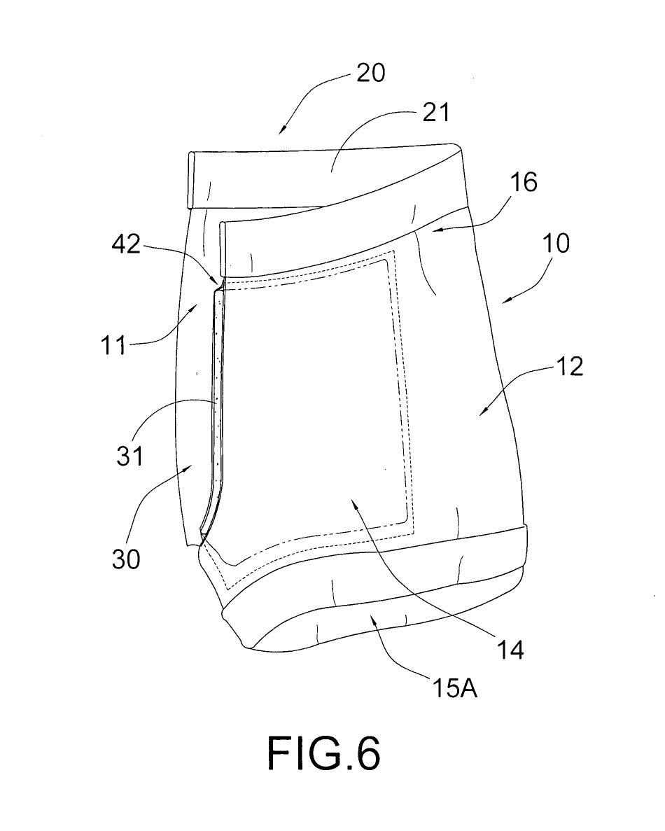

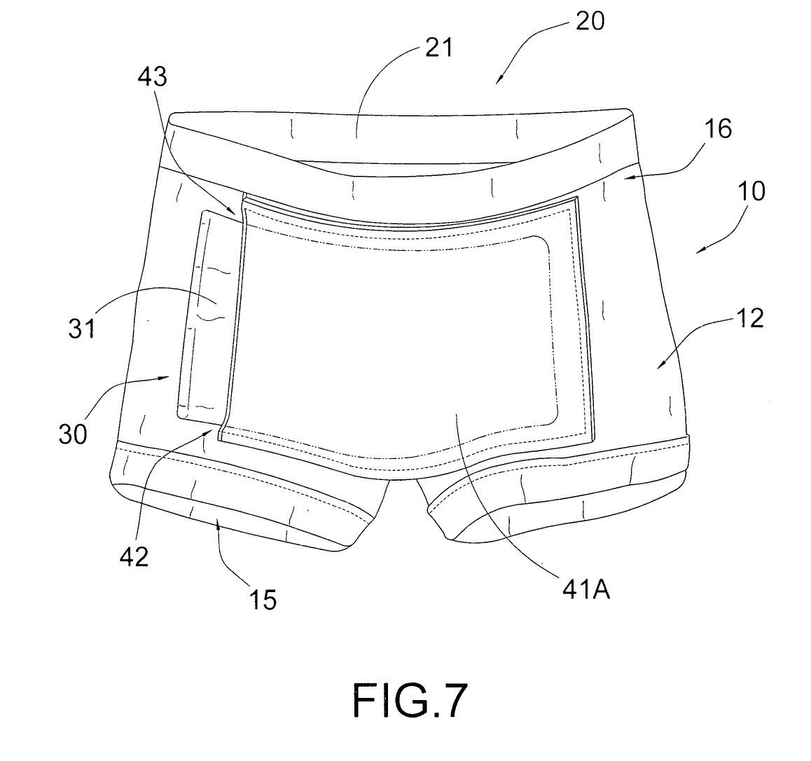

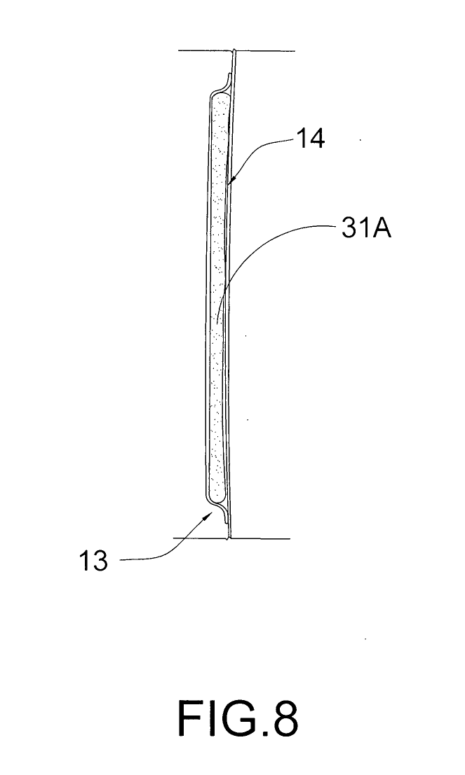

Referring to FIG. 6 to FIG. 8 of the drawings, a urine-free underwear such as an underwear garment for wearing on lower torso of a wearer according to a second preferred embodiment of the present invention is illustrated, in which the underwear garment comprises an underwear fabric 10A, a waistband 20A, and a liquid controlling layer 30A which is capable of preventing liquid from reaching out thereof and coming into contact with the underwear fabric 10A or the external clothing of the wearer.

The underwear fabric 10A defines a buttock covering section 11A, a hip covering section 12A, a bottom covering section 13A, a front covering section 14A, and a pair of leg holes 15A for allowing two legs of the wearer to pass therethrough. On the other hand, the waistband 20A is provided on a top edge portion 16A of the underwear fabric 10A.

The liquid controlling layer 30A is provided on an inner side of the underwear fabric 10A on at least one of the bottom covering section 13A and the front covering section 14A of the underwear fabric 10A for absorbing liquid coming into contact with the liquid controlling layer 30A. The underwear garment further comprises means for securing the liquid controlling layer 30A onto the inner side of the underwear fabric 10A (referred to securing means below). Note that the front covering section 14A and the bottom covering section 13A are genital section of the underwear fabric as defined above.

According to the second preferred embodiment of the present invention, the underwear fabric 10A is made of regular fabric material used for conventional underwear garment, such as an underwear pant for men. The underwear garment can also be embodied as boxers or any other forms of underwear for wearing on a lower torso of a wearer, who can be a man or a woman. The second preferred embodiment of the present invention is to design the underwear garment for a man as a wearer.

The means for securing the liquid controlling layer 30A onto the inner side of the underwear fabric 10A comprises an inner pocket layer 41A attached on the front covering section 14A of the underwear fabric 10A to define a receiving pocket 42A between the inner pocket layer 41A and the front covering section 14A of the underwear fabric 10A. The inner pocket layer 41A is shaped and sized to correspond to a shape and size of the front covering section 14A so that the inner pocket layer 41A is in direct contact with the genital (i.e. the penis) of the wearer. The liquid controlling layer 30A is fittedly received into the receiving pocket 42A so that when liquid, such as urine, comes into contact with the inner pocket layer 41A, the liquid will penetrate through the inner pocket layer 41A and will be in contact with the liquid controlling layer 30A. The liquid controlling layer 30A will then trap the liquid (i.e. the urine) within the liquid controlling layer 30A so as to keep the inner pocket layer 41A dry and maintain good hygienic condition for the wearer.

The inner pocket layer 41A also defines an opening 43A communicating the receiving pocket 18A and an exterior of that receiving pocket 42A. The purpose of this opening 43A is to allow the wearer of the underwear garment to remove the liquid controlling layer 30A from the receiving pocket 42A and replace the used liquid controlling layer 30A by a new liquid controlling layer 30A. The wearer will put the new liquid controlling layer 30A into the receiving pocket 42A. It is worth mentioning that the opening 43A may be selectively opened and so as to selectively allow the wearer to gain access to the receiving pocket 42A for replacing the liquid controlling layer 30A.

The waistband 20A comprises an elastic element 21A peripherally formed along a top edge portion of the underwear fabric 10A for allowing the waistband 20A to be fittedly worn onto the wearer's lower torso.

The liquid controlling layer 30A preferably comprises a liquid absorbing layer comprising a fabric layer 31A consisting of a predetermined kind of fibers, and a predetermined amount of absorbent chemicals formed on the fabric layer 31A for absorbing liquid (such as urine of the wearer) coming into contact with the liquid controlling layer 30A through the inner pocket layer 41A.

The operation of the present invention is as follows: a wearer may put the liquid controlling layer 30A into the receiving pocket 42A of the securing means and wear on the underwear garment. If the wearer has difficulty to control urine discharge due to some diseases or other reasons, the urine coming out from the wearer's penis will pass through the inner pocket layer 41A and come into contact with the liquid controlling layer 30A. The liquid controlling layer 30A will then absorb the urine and keep it within the liquid controlling layer 30A. As a result, the inner pocket layer 41A of the securing means and all other portions of the underwear fabric 10A will be kept dry and the maximum amount of hygienic condition will be maintained. After use, the wearer may replace the water absorbing layer 30A with an unused one. Note that the used liquid controlling layer 30A can be disposed or washed for reuse. The exact configuration of liquid controlling layer 30A (i.e. whether it is meant to be disposable or washable for reuse) depends on particular manufacturing and marketing conditions of the present invention.

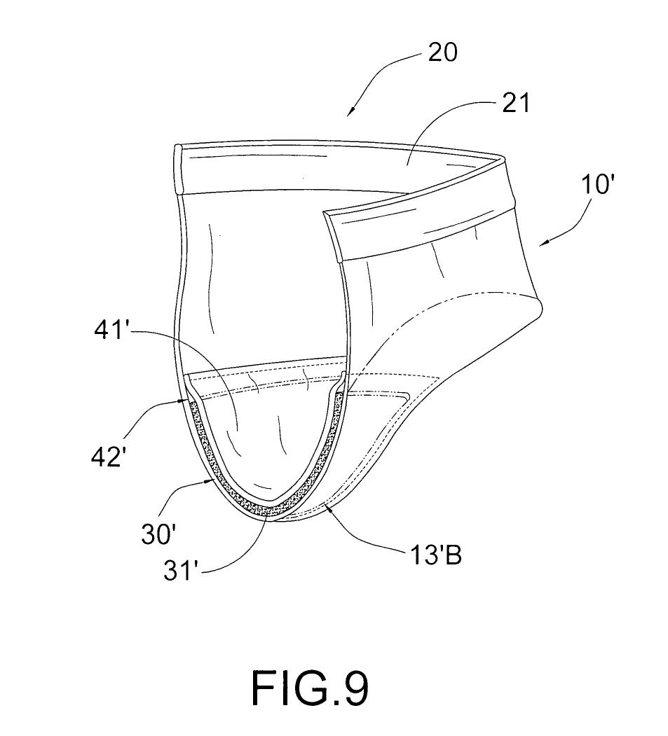

Referring to FIG. 9 of the drawings, a first alternative mode of the urine-free underwear according to the second preferred embodiment of the present invention is illustrated. The first alternative mode is similar to the second preferred embodiment described above, except that the underwear garment is meant to be worn by a female wearer so that the positions of the liquid controlling layer 30B and the securing means are different.

According to the first alternative mode, the means for securing the liquid controlling layer 30B onto the inner side of the underwear fabric 10B comprises an inner pocket layer 41B attached on the bottom covering section 13B of the underwear fabric 10B to define a receiving pocket 42B between the inner pocket layer 41B and the bottom covering section 14B of the underwear fabric 10B. The inner pocket layer 41B is shaped and sized to correspond to a shape and size of the bottom covering section 13B for fitting female urethra structure. The fabric layer 31B of the liquid controlling layer 30B is fittedly received into the receiving pocket 42B so that when liquid, such as urine, comes into contact with the inner pocket layer 41B, the liquid will penetrate through the inner pocket layer 41B and will be in contact with the liquid controlling layer 30B. The liquid controlling layer 30B will then trap the liquid (i.e. the urine) within the liquid controlling layer 30B so as to keep the inner pocket layer 41B dry and maintain good hygienic condition for the wearer. The waistband 20B and the elastic elements 21B are kept to the same as in the second preferred embodiment mentioned above.

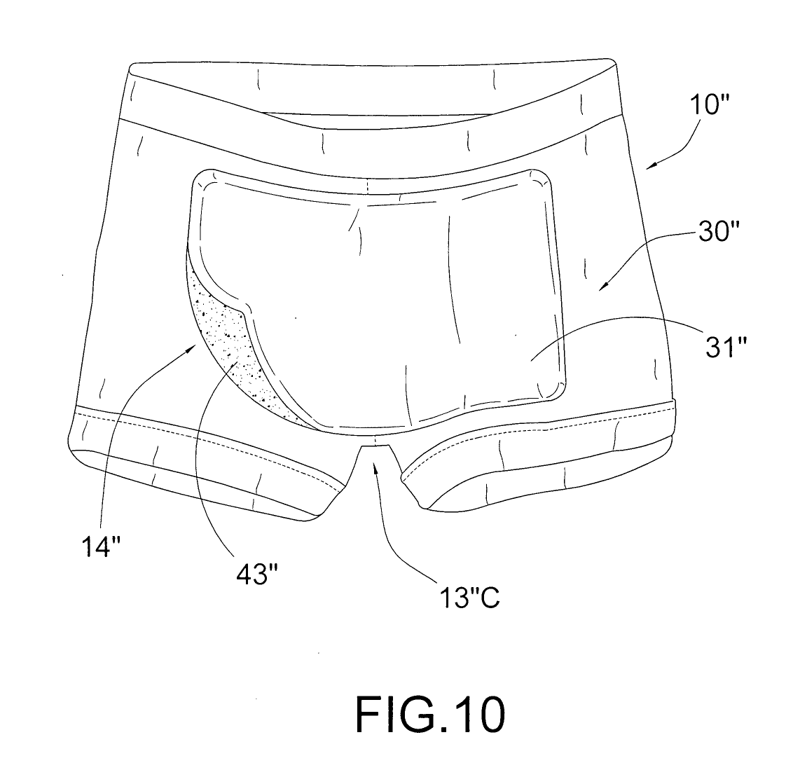

Referring to FIG. 10 of the drawings, a second alternative mode of the urine-free underwear according to the second preferred embodiment of the present invention is illustrated. The second alternative mode is similar to the preferred embodiment except the means for securing the liquid controlling layer 30C onto the inner side of the underwear fabric 10C. According to the second alternative mode, the securing means comprises an adhesive layer 43C applied on the liquid controlling layer 30C so as to allow the liquid controlling layer 30C to be directly and detachably attached onto the front covering section 14C of the underwear fabric 10C. In other words, the liquid controlling layer 30C is not protected by the receiving pocket 42A. Rather, the liquid controlling layer 30C is directly but detachably attached onto the inner side of the front covering section 14C of the underwear fabric 10C. When the urine-free underwear according to this second alternative mode is for female's use, the liquid controlling layer 30C is detachably attached onto the bottom covering section 13C (13A) of the underwear fabric 10C so as to accommodate female's biological structure.

As a result, when the liquid controlling layer 30C has been used for absorbing urine, the user can easily and conveniently replace the used liquid controlling layer 30C with an unused one. Moreover, the user may remove the liquid controlling layer 30C before washing the underwear garment.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. An underwear for wearing on lower torso of a wearer, comprising:
an underwear fabric defining a genital covering section;
a waistband provided on a top edge portion of said underwear fabric;
a liquid controlling layer provided on an inner side of said underwear fabric on genital covering section of said underwear fabric near a genital of said wearer for blocking liquid coming into contact with said genital covering section of said underwear fabric; and
means for securing said liquid controlling layer onto said inner side of said underwear fabric.

2. The underwear, as recited in claim 1, wherein said means for securing said liquid controlling layer onto said inner side of said underwear fabric comprises an inner pocket layer attached on said front covering section of said underwear fabric to define a receiving pocket between said inner pocket layer and said genital covering section of said underwear fabric, wherein said inner pocket layer is shaped and sized to correspond to a shape and size of said genital covering section so that said inner pocket layer is in direct contact with said genital of said wearer.

3. The underwear, as recited in claim 2, wherein said waistband comprises an elastic element peripherally formed along a top edge portion of said underwear fabric for allowing said waistband to be fittedly worn onto said wearer's lower torso.

4. The underwear, as recited in claim 3, wherein said waistband comprises an elastic element peripherally formed along a top edge portion of said underwear fabric for allowing said waistband to be fittedly worn onto said wearer's lower torso.

5. The underwear, as recited in claim 2, wherein said genital covering section is one of a front covering section of said underwear fabric and a bottom covering section of said underwear fabric so as to fit male's and female's use of said underwear fabric respectively.

6. The underwear, as recited in claim 4, wherein said genital covering section is one of a front covering section of said underwear fabric and a bottom covering section of said underwear fabric so as to fit male's and female's use of said underwear fabric respectively.

7. The underwear, as recited in claim 5, wherein said liquid controlling layer is a liquid impermeable layer which has a physical property of blocking liquid from penetrating through said liquid blocking layer.

8. The underwear, as recited in claim 6, wherein said liquid controlling layer is a liquid impermeable layer which has a physical property of blocking liquid from penetrating through said liquid blocking layer.

9. The underwear, as recited in claim 5, wherein said liquid controlling layer is arranged to absorb liquid coming into contact with said liquid controlling layer, wherein said liquid controlling layer comprises a fabric layer comprising a predetermined amount of fibers, and a predetermined amount of absorbent chemicals formed on said fabric layer for absorbing liquid coming into contact with said liquid controlling layer.

10. The underwear, as recited in claim 6, wherein said liquid controlling layer is arranged to absorb liquid coming into contact with said liquid controlling layer, wherein said liquid controlling layer comprises a fabric layer comprising a predetermined amount of fibers, and a predetermined amount of absorbent chemicals formed on said fabric layer for absorbing liquid coming into contact with said liquid controlling layer.

11. The underwear, as recited in claim 1, wherein said means for securing said liquid controlling layer onto said inner side of said underwear fabric comprises an adhesive layer applied on said liquid controlling layer so as to allow said liquid controlling layer to be directly and detachably attached onto said genital covering section of said underwear fabric.

12. The underwear, as recited in claim 11, wherein said waistband comprises an elastic element peripherally formed along a top edge portion of said underwear fabric for allowing said waistband to be fittedly worn onto said wearer's lower torso.

13. The underwear, as recited in claim 11, wherein said genital is one of a front covering section of said underwear fabric and a bottom covering section of said underwear fabric so as to fit male's and female's use of said underwear fabric respectively.

14. The underwear, as recited in claim 12, wherein said genital is one of a front covering section of said underwear fabric and a bottom covering section of said underwear fabric so as to fit male's and female's use of said underwear fabric respectively.

15. The underwear, as recited in claim 11, wherein said liquid controlling layer is a liquid impermeable layer which has a physical property of blocking liquid from penetrating through said liquid blocking layer.

16. The underwear, as recited in claim 13, wherein said liquid controlling layer is a liquid impermeable layer which has a physical property of blocking liquid from penetrating through said liquid blocking layer.

17. The underwear, as recited in claim 14, wherein said liquid controlling layer is a liquid impermeable layer which has a physical property of blocking liquid from penetrating through said liquid blocking layer.

18. The underwear, as recited in claim 11, wherein said liquid controlling layer is arranged to absorb liquid coming into contact with said liquid controlling layer, wherein said liquid controlling layer comprises a fabric layer comprising a predetermined amount of fibers, and a predetermined amount of absorbent chemicals formed on said fabric layer for absorbing liquid coming into contact with said liquid controlling layer.

19. The underwear, as recited in claim 13, wherein said liquid controlling layer is arranged to absorb liquid coming into contact with said liquid controlling layer, wherein said liquid controlling layer comprises a fabric layer comprising a predetermined amount of fibers, and a predetermined amount of absorbent chemicals formed on said fabric layer for absorbing liquid coming into contact with said liquid controlling layer.

20. The underwear, as recited in claim 14, wherein said liquid controlling layer is arranged to absorb liquid coming into contact with said liquid controlling layer, wherein said liquid controlling layer comprises a fabric layer comprising a predetermined amount of fibers, and a predetermined amount of absorbent chemicals formed on said fabric layer for absorbing liquid coming into contact with said liquid controlling layer.
